(19) United States
(12) Patent Application Publication
(10) Pub. No.: US 2015/0164919 A1
(43) Pub. Date: Jun. 18, 2015

(54) SKIN CLEANSING COMPOSITION WITH A DEPOSITION COMPONENT

(71) Applicant: The Dial Corporation, Scottsdale, AZ (US)

(72) Inventors: Chris Luciow, Phoenix, AZ (US); Terannie Vazquez Alvarez, Gilbert, AZ (US)

(73) Assignee: The Dial Corporation, Scottsdale, AZ (US)

(21) Appl. No.: 14/108,721

(22) Filed: Dec. 17, 2013

(51) Int. Cl. A61K 31/60; A61K 9/00

(52) U.S. Cl. CPC A61K 31/60; A61K 9/0014

(57) ABSTRACT

Materials and apparatus are provided for a skin cleaning composition with a cationic deposition component. The skin cleaning composition includes a keratolytic skin peeling ingredient. The skin cleaning composition further includes at least one surfactant. The skin cleaning composition further includes a cationic deposition component to enhance deposition of the keratolytic skin peeling ingredient onto the skin.

### FIELD OF THE INVENTION

The present invention relates to a composition to enhance the efficacy of acne treatment, and more particularly relates to a cleansing composition to increase deposition of keratolytic skin peeling ingredients onto the skin.

### BACKGROUND OF THE INVENTION

Acne is a common problem for both teenagers and adults. It may be characterized by inflammation of the sebaceous glands and may result in pustules on the skin. Acne cleansing formulations may use salicyclic acid in a formulation with a fairly acidic pH, which may result in the available acne cleansing formulations being harsh to the skin. Such harshness may result in discomfort, skin irritation, and extended use could cause the acne to worsen over time.

Accordingly, it is desirable to have an acne cleansing composition that is mild and that does not irritate the skin. In addition, it is desirable to have an acne cleansing composition that is capable of depositing the active ingredient onto the skin. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A skin cleansing composition with a cationic deposition component includes a keratolytic skin peeling ingredient. The composition also includes at least one surfactant. The composition also includes a cationic deposition component to enhance deposition of the keratolytic skin peeling ingredient onto the skin.

A skin care product with a cationic deposition component includes a liquid skin cleansing composition. The liquid skin cleansing composition includes a keratolytic skin peeling ingredient. The skin cleansing composition also includes at least one surfactant. The skin cleansing composition also includes a cationic deposition component to enhance deposition of the keratolytic skin peeling ingredient onto the skin. The skin cleansing composition also includes an aqueous medium to distribute the keratolytic skin peeling ingredient, at least one surfactant, cationic deposition component, or combinations thereof. The skin care product also includes a container to dispense the liquid skin cleaning composition.

A skin cleansing composition with a deposition component includes a keratolytic skin peeling ingredient. The composition also includes at least one surfactant. The composition also includes a cationic polymer containing quaternary amines. The composition also includes a number of cationic conditioning agents, nonionic condition agents, or combinations thereof.

### BRIEF DESCRIPTION OF THE DRAWINGS

The present invention will hereinafter be described in conjunction with the following drawing figures, wherein like numerals denote like elements, and

FIG. 1 is a diagram of an exemplary container dispensing a skin cleansing composition according to the principles described herein.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Acne may be caused by the oily secretions around the hair follicles combining with excess skin cells to form a plug. These plugs may be the core cause of acne. When the area beneath the plug contains bacteria, a pimple may be produced at that site. When the oily secretions continue underneath the plug, a pimple may be formed at that site. Hormones may be responsible for increased production of the oily secretion around hair follicles, which may result in increased acne. Acne may develop in both teenagers and adults of both sexes, and may be a considerable source of embarrassment for those who experience it. Such blemishes may be considered unsightly, and the person experiencing acne may go to some length in order to both conceal existing acne and decrease the risk of future acne.

One such measure to reduce the risk of future acne may be a wash-off formulation to treat the underlying causes of acne. However, such wash-off formulations may be a fairly harsh treatment at acidic pH. For example, these wash-off formulations may irritate the skin and be uncomfortable for a consumer and may also include unsightly results such as rashes, etc.

Accordingly, the principles described herein provide a cationic deposition component to deposit the keratolytic skin peeling ingredient onto the skin, which may allow for more extended contact and more effective action in a milder formulation. A keratolytic skin peeling ingredient and surfactants may also be included in the cleansing composition for the treatment of acne.

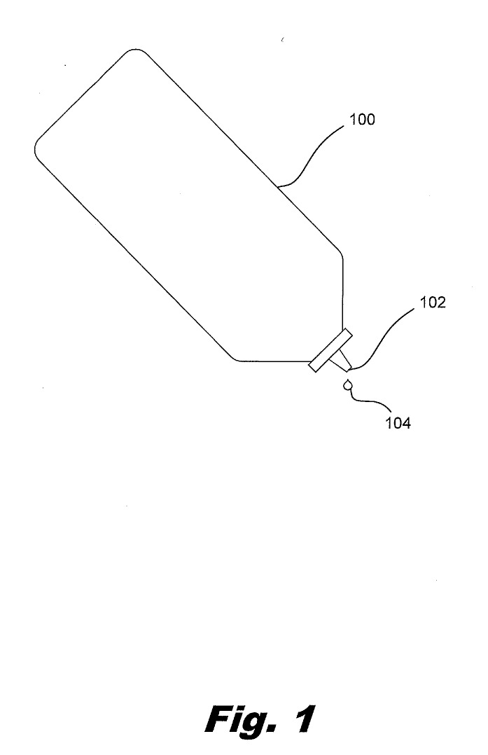

Turning now to the figures, FIG. 1 is a diagram of an exemplary container (100) dispensing a skin cleansing composition (104) according to the principles described herein? The container (100), equipped with an opening (102), may contain a skin cleansing composition (104) for the treatment or prevention of acne. Such acne may be present on the face, hands, arms, or other areas of the skin. Accordingly, the opening (102) may allow deposition of the skin cleansing composition (104) on an area of the skin. In this example, the skin cleansing composition (104) may be held within a container (100) that has an opening (102) which may allow the skin cleansing composition (104) to flow out of the container (100), whereby allowing it to be accessed by the user. The skin cleansing composition (104) contained within the container (100) may be applied to the skin of the user for the treatment or prevention of acne, and may be subsequently rinsed off by application of water to the treated area(s). The container shown in FIG. 1 (100) is exemplary, and does not represent all types of containers or all types of dispensers which may be used to dispense the liquid cleaning composition (104). Other types of dispensers may include a plunger which dispenses the skin cleaning composition (104) when depressed towards the container (100).

The skin cleansing composition (104) may include a keratolytic skin peeling ingredient. The skin cleansing composition may comprise between 0.5 and 3.0 weight percent keratolytic skin peeling ingredient. The keratolytic skin peeling ingredient may be an agent that may thin skin layers that are thicker than desirable. Accordingly, the keratolytic skin peeling ingredient may slough off an outer layer of skin. The keratolytic skin peeling ingredient may prevent the formation of a plug around the hair follicles, which may allow the oily secretion to lubricate the hair follicle rather than accumulate underneath such a plug. Accordingly, by promoting the sloughing off of excess layers of skin, a keratolytic skin peeling ingredient may remove one of the elements that is responsible for the formation of acne, which may in turn result in a decrease in the amount of acne experienced by an individual. In some examples, the keratolytic skin peeling ingredient may be salycyclic acid. While specific reference is made to salycyclic acid, any type of keratolytic skin peeling ingredient may be used to aid in sloughing off of skin layers. For example, the skin cleansing composition (104) may include a number of organic acids as a skin peeling ingredient.

The skin cleansing composition (104) may include at least one surfactant. The skin cleansing composition may comprise between 5 and 20 weight percent of a number of surfactants. A surfactant may have a hydrophobic end and a hydrophilic end. The hydrophobic end may allow the surfactant to interact with uncharged molecules, such as oils. The hydrophobic end may be a hydrocarbon, which may be either linear, branched, cyclic or aromatic. The hydrophilic end may facilitate the interaction of the molecule with charged or polar molecules, such as water. The hydrophilic end may be used to classify surfactants, which may be anionic, cationic, nonionic, or zwitterionic. Anionic surfactants may have a negatively charged hydrophilic end, which may be present as a sulfate, sulfonate, carboxylate or the like; anionic surfactants may be sensitive to water hardness. Cationic surfactants may be those that have a positively charged hydrophilic end, such as a quaternary amine. Nonionic surfactants may have a hydrophilic end which may be charge neutral, such as an ethoxylate or poly-ol; such surfactants may not be sensitive to water hardness. Zwitterionic or amphoteric surfactants may have both a positive and negative charge on their hydrophilic ends, such as amine oxides. In some examples, the at least one surfactant may include sodium laureth sulfate, sodium lauryl sulfate, lauramidopropyl betaine, lauryl betaine, cocamidopropyl betaine, or combinations thereof. While specific reference has been made to certain surfactants, the at least one surfactant in the skin cleansing composition (104) may include any type, combination or mixture of surfactants. For example, the surfactants may include a blend of cationic and zwitterionic surfactants.

The skin cleansing composition (104) may include a cationic deposition component to enhance deposition of the keratolytic skin peeling ingredient onto the skin. The skin cleansing composition may comprise between 0.05 and 3.0 weight percent of such a cationic deposition component. The deposition component may provide the skin peeling ingredient additional time to act, enabling more effective sloughing off of excess skin and may result in improved prevention and treatment of acne. The inclusion of a cationic deposition component may allow the formulation to be milder by allowing a more neutral pH value of the skin cleansing composition (104).

The cationic deposition component may be designated as cationic because it has a net positive charge; the potential inclusion of non-charged elements in the cationic deposition component does not alter its designation as cationic, so long as the deposition component retains a net positive charge.

The cationic deposition component may include a number of antistatic agents, which may act by adsorbing onto the skin surface whereby changing its surface characteristics. Such changes to the surface characteristics of the skin surface may result in enhanced deposition of the keratolytic skin peeling ingredient. Antistatic agents may also act by altering the electrostatic properties of cosmetic raw materials or the skin, and may function by reducing the tendency of either component to acquire an electrical charge. One example of an antistatic agent is a cationic polymer. More specifically, the deposition component may include a cationic polymer that contains quaternary amines in a monomeric unit. Examples of such cationic polymers include Polyquaternium-7 and Polyquaternium-10.

Another example of an antistatic agent is a conditioning agent. In other words, the cationic deposition component may include a conditioning agent. Such conditioning agents may be cationic, nonionic or zwitterionic. For example, the skin cleansing composition (104) may include isostearamidopropyl morpholine lactate as a nonionic conditioning agent. The skin cleansing composition (104) may include cocamidopropyl PG_Dimonium Chloride as a cationic conditioning agent. The addition of conditioning agents, which may be either cationic, nonionic, or a combination thereof, allows for a milder formulation which does not detract from the ability of the cationic polymer to enhance the deposition of the keratolytic skin peeling ingredient onto the skin.

The cationic deposition component may deposit the keratolytic skin peeling ingredient directly onto the skin. Such a deposition directly onto the skin might not involve the encapsulation of the keratolytic skin peeling ingredient. Deposition of the keratolytic skin peeling ingredient directly onto the skin may allow more direct hydrogen exchange between the keratolytic skin peeling ingredient and its local environment, which may result in more direct or expedited activation of the keratolytic skin peeling ingredient.

The skin cleansing composition (104) may be configured to combat acne. Accordingly, the cationic deposition component may be configured to deposit the keratolytic skin peeling ingredient onto the skin. For example, the skin cleansing composition (104) may be included in a skin cleaning product that is to be used as a shampoo composition, a body wash composition, or a face wash composition. The cationic deposition component may enable an increased time for the keratolytic skin peeling ingredient to act over typical wash-off formulations. The deposition of the skin peeling ingredient onto the skin may allow it to act for a much longer time, which may decrease the amount of product to effectively treat acne. Additionally, this may enable a product to be effective at a higher pH, whereby enabling a milder formulation.

In some examples, the skin cleansing composition (104) may include other ingredients such as citric acid, fragrance, dye, thickening agents, components to improve the lather and feel of the skin cleansing composition (104) which may increase consumer appeal. Moreover, in some examples, the skin cleansing composition may be a rinse-off formulation. In some examples, the skin cleansing composition (104) may have a pH between 4.0 and 6.0 to increase the mildness of the skin cleansing composition (104).

Table (1) illustrates an exemplary composition of the skin cleansing composition (104) described herein. The composition in Table (1) may be suitable for use as a body wash for the treatment or prevention of acne. In the disclosed formulation, salicyclic acid may be the keratolytic skin peeling ingredient used, and two surfactants may be employed. Polyquaternium-10 may be used in the cationic deposition component as a cationic polymer. Additionally, cocamidopropyl PG-dimonium chloride and isostearamidopropyl morpholine lactate may also be used in the cationic deposition component as a cationic conditioning agent and a nonionic conditioning agent, respectively. Thickening agents, dyes, chelating agents, fragrances and other components may also be included. The pH of the resultant skin cleansing composition (104) may be between 4.0 and 5.0, and the viscosity may range from 5,000 to 35,000 centipoise.

TABLE (1)

| | % of Composition |
|---|---|
| Ingredient | (by weight) |
| Water | 82.57 |
| Sodium Laureth Sulfate | 6.50 |
| Salicyclic Acid | 2.00 |
| Glycerin | 1.25 |
| Cocamidopropyl Betaine | 5.18 |
| Isostearamidopropyl Morpholine Lactate | 0.10 |
| Cocamidopropyl PG-Dimonium Chloride | 0.35 |
| Citric Acid (Anhydrous) | 0.010 |
| Sodium Hydroxide | 0.37 |
| Polyquaternium-10 | 0.30 |
| Tetrasodium EDTA | 0.02 |
| Fragrance | 0.89 |
| Water, glycerin, Citrus Aurantium Dulcis | 0.050 |
| (Orange) Juice, Citrus Paradisi (Grapefruit) | |
| juice, Passiflora Edulis Fruit Juice | |
| Red 4 | 0.000083 |
| Yellow 5 | 0.000180 |
| PEG-200 Hydrogenated Glyceryl Palmate, | 0.40 |
| PEG-7 Glyceryl Cocoate | |
| Sodium Chloride | 0.010 |

Table (2) illustrates another example composition of the skin cleansing composition (104) described herein. The composition of Table (2) may be suitable as a face wash for the treatment or prevention of acne. As with the formulation in Table (1), salicyclic acid may be used as the keratolytic skin peeling ingredient, and two surfactants may be employed, including sodium laureth sulfate and cocamidopropyl betaine. The cationic deposition component is again comprised of polyquaternium-10 as a cationic polymer, cocamidopropyl PG-dimonium chloride as a cationic conditioning agent and isostearamidopropyl morpholine lactate as a nonionic conditioning agent. As with the formulation in Table (1), thickening agents, dyes, fragrances and other components may also be included. The pH of the resultant skin cleansing composition may be between 4.0 and 5.0, and the viscosity may range from 2,000 to 22,000 centipoise.

TABLE (2)

| | % of Composition |
|---|---|
| Ingredient | (by weight) |
| Water | 83.99 |
| Sodium Laureth Sulfate | 6.50 |
| Salicyclic Acid | 2.00 |
| Glycerin | 1.25 |
| Cocamidopropyl Betaine | 4.40 |
| Isostearamidopropyl Morpholine Lactate | 0.10 |
| Cocamidopropyl PG-Dimonium Chloride | 0.35 |
| Citric Acid (Anhydrous) | 0.010 |
| Sodium Hydroxide | 0.37 |
| Polyquaternium-10 | 0.30 |
| Tetrasodium EDTA | 0.02 |
| Fragrance | 0.35 |
| Water, glycerin, Citrus Aurantium Dulcis | 0.050 |
| (Orange) Juice,Citrus Paradisi (Grapefruit) | |
| juice, Passiflora Edulis Fruit Juice | |
| Red 4 | 0.000083 |
| Yellow 5 | 0.000180 |
| PEG-200 Hydrogenated Glyceryl Palmate, | 0.30 |
| PEG-7 Glyceryl Cocoate | |
| Sodium Chloride | 0.010 |

As keratolytic skin peeling ingredients may be acids, for example salicyclic acid or a number of organic acids, and the protonated form may be active while the deprotonated form may be inactive, the pKₐ of certain keratolytic skin peeling ingredients may determine the efficiency with which that keratolytic skin peeling ingredient functions at a given pH. As acidic pH may be harsh or irritate the skin, it is desirable that the pH of the skin cleansing formulation be close to neutral while also maintaining the efficacy of the keratolytic skin peeling ingredient. By including a cationic deposition component, the time in which the keratolytic skin peeling ingredient is in contact with the skin may be increased, which may decrease the significance of the fraction of an acidic keratolytic skin peeling ingredient that is protonated at the pH of the skin cleansing formulation. Thus the cationic deposition component may allow for the skin cleansing formulation to be at a milder pH while maintaining efficacy in the treatment of acne.

Clinical tests may show that a skin cleansing composition (104) is effective in reducing acne, and may also show whether or not a skin cleansing composition (104) is gentle enough for regular use. Such a clinical test may involve a baseline assessment of acne, which may be accomplished by counting lesions on a specified application area, which may be on either the face or the back/shoulders of the test subject. Subjects in a clinical test may be randomly divided into a treatment group and a control group; the treatment group may receive the skin cleansing composition (104), and the control group may receive a skin cleansing composition of known properties. Subjects may be instructed to wash the test site two times per day with the provided composition. Subjects may agree not to take actions which may affect the results of the study, for example application of cosmetics or toiletry products to the test site. During the course of a ten-day treatment with the skin cleansing composition (104) or a control composition, lesions may be counted at regular intervals, for example at days 3, 7 and 10, and these measurements may be compared to the baseline value. The subject's skin may also be visually assessed for dryness and erythema at each visit, which may be scored from 0 to 4, using integer values. Dryness and erythema may be assessed separately. Such a clinical study may be done on a variety of individuals, which may demonstrate both tolerance and efficacy on a variety of skin types.

Table (3) may show data from a clinical test of the skin cleansing composition (104), which may show that it is effective in reducing acne. Table (3) may also present data from a control composition alongside the data for the skin cleansing composition (104). The data in Table (3) may indicate the total number of acne lesions at baseline, after 3 days of treatment, after 7 days of treatment, and after 10 days of treatment with either the skin cleansing composition (104) or the control composition. The total number of acne lesions presented in Table (3) may include both inflamed and non-inflamed lesions. The total number of acne lesions presented in Table (3) may be the average number of acne lesions per subject in the clinical test group of 90 subjects, 45 of which received the skin cleansing composition (104), and 45 of which received the control composition. Table (3) may present data for application of either the skin cleansing composition (104) to the back/shoulders, which may be indicated as (back), and application to the face, which may be indicated as (face).

TABLE (3)

| Treatment | Baseline | Day 3 | Day 7 | Day 10 |
|---|---|---|---|---|
| Control (back) | 21.00 | 21.70 | 21.13 | 21.91 |
| Skin Cleansing | 19.24 | 16.33 | 13.13 | 12.53 |
| Composition | | | | |
| (104) (back) | | | | |
| Control (face) | 30.78 | 32.80 | 32.16 | 33.49 |
| Skin Cleansing | 29.51 | 26.56 | 24.80 | 23.53 |
| Composition | | | | |
| (104) (face) | | | | |

Table (4) may show data from a clinical test of the skin cleansing composition (104), which may show that the skin cleansing composition (104) is well tolerated. Table (4) may also present data from a control composition alongside the data for the skin cleansing composition (104). The data in Table (4) may be based on a clinical test group of 90 subjects, 45 of which received the skin cleansing composition (104), and 45 of which received a control composition. Table (4) may present dryness and erythema for each treatment, which may be assessed separately, and may be scored from 0 to 4, using integer values for each subject. In such a scoring scale, 0 may be used to indicate no visible dryness or erythema; 1 may be used to indicate very light or slight visible dryness or erythema; 2 may be used to indicate light or mild visible dryness or erythema; 3 may be used to indicate moderate diffuse or dense visible dryness or erythema; 4 may be used to indicate prominent and dense visible dryness or erythema. The values presented in Table (4) may be the average for the indicated test group. Table (4) may present values for both application of the skin cleansing composition (104) or the control composition to the back, which may be indicated by (back), and the face, which may be indicated by (face). Visible dryness and erythema may be assessed separately, and Table (4) may present values as ‘dryness (erythema);’ these are presented alongside one another in order to allow for ready comparison between the effects of each treatment on dryness and the effects of the same treatment on erythema.

TABLE (4)

| Treatment | Baseline | Day 3 | Day 7 | Day 10 |
|---|---|---|---|---|
| Control (back) | 0.00 (0.00) | 0.16 (0.00) | 0.09 (0.00) | 0.04 (0.00) |
| Skin Cleansing | 0.00 (0.00) | 0.16 (0.00) | 0.11 (0.00) | 0.00 (0.00) |
| Composition | | | | |
| (104) (back) | | | | |
| Control (face) | 0.00 (0.00) | 0.18 (0.00) | 0.11 (0.00) | 0.09 (0.00) |
| Skin Cleansing | 0.00 (0.00) | 0.24 (0.00) | 0.38 (0.00 | 0.09 (0.02) |
| Composition | | | | |
| (104) (face) | | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A skin cleansing composition with a cationic deposition component, comprising:
a keratolytic skin peeling ingredient;
at least one surfactant; and
a cationic deposition component to enhance deposition of the keratolytic skin peeling ingredient onto the skin.

2. The composition of claim 1, wherein the keratolytic skin peeling ingredient is salicyclic acid.

3. The composition of claim 1, wherein the at least one surfactant comprises sodium laureth sulfate, sodium lauryl sulfate, lauramidopropyl betaine, lauryl betaine, cocamidopropyl betaine, or combinations thereof.

4. The composition of claim 1, wherein the cationic deposition component comprises a number of cationic polymers containing quaternary amines in the monomeric unit.

5. The composition of claim 4, wherein the number of cationic polymers comprise Polyquaternium-10, Polyquaternium-7, or combinations thereof.

6. The composition of claim 1, in which the cationic deposition component further comprises a nonionic conditioning agent.

7. The composition of claim 6, wherein the nonionic conditioning agent is isostearamidopropyl morpholine lactate.

8. The composition of claim 1, in which the cationic deposition component further comprises a cationic conditioning agent.

9. The composition of claim 8, in which the cationic conditioning agent is Cocamidopropyl PG-Dimonium Chloride.

10. A skin care product to deposit one or more keratolytic skin peeling ingredients onto the skin, comprising:
a skin cleansing composition, wherein the skin cleansing composition includes:
a keratolytic skin peeling ingredient; and
at least one surfactant; and
a cationic deposition component to enhance deposition of the keratolytic skin peeling ingredient onto the skin; and
an aqueous medium in which the above components are distributed; and
a container to dispense the skin cleansing composition.

11. The product of claim 10, wherein the keratolytic skin peeling ingredient is salicyclic acid.

12. The product of claim 10, wherein the cationic deposition component comprises of cationic polymer containing quaternary amines.

13. The product of claim 10, wherein the cationic deposition component is configured to deposit the keratolytic skin peeling ingredient onto the skin.

14. The product of claim 10, wherein the aqueous medium further comprises citric acid, fragrance, dye, a thickening agent, or combinations thereof.

15. The product of claim 10, wherein the skin cleaning composition has a pH between 4.0 and 6.0.

16. The product of claim 10, in which the cationic deposition component comprises, nonionic conditioning agents, cationic conditioning agents, or combinations thereof.

17. The product of claim 10, wherein the skin cleaning composition is a rinse-off formulation.

18. The product of claim 10, wherein the skin cleaning composition is configured to combat acne.

19. The product of claim 10, wherein the skin cleaning composition is formulated for application to the face.

20. A skin cleansing composition with a deposition component, comprising:
between 0.1% and 5% by weight of a keratolytic skin peeling ingredient; and
between 5% and 20% by weight of at least one surfactant; and
between 0.01% and 2% by weight of a cationic polymer containing quaternary amines; and
between 0.01% and 5% by weight of a number of conditioning agents, which may be either cationic, nonionic, or combinations thereof.
